(19) United States
(12) Patent Application Publication
(10) Pub. No.: US 2010/0305519 A1
(43) Pub. Date: Dec. 2, 2010

(54) CANNULA HAVING AN OVERLAPPING CANNULA FEATURE AND NOTCH FEATURE

(75) Inventors: Austin Jason McKinnon, Herriman, UT (US); Marty L. Stout, South Weber, UT (US)

Correspondence Address: David W. Highet, VP & Chief IP Counsel;Becton, Dickinson and Company, (Kirton & McConkie), 1 Becton Drive, MC 110, Franklin Lakes, NJ, 07417-1880, US

(73) Assignee: Becton, Dickinson and Company, Franklin Lakes, NJ (US)

(21) Appl. No.: 12/476,961

(22) Filed: Jun. 2, 2009

(51) Int. Cl. A61M 25/06; B23P 11/00; A61M 25/00

(52) U.S. Cl. 604/272; 29/428; 604/523

(57) ABSTRACT

A cannula having a notch feature and a cannula feature that at least partially overlap each other is described herein. Generally, the cannula comprises a tubular shaft with a substantially constant outer diameter. The cannula feature comprises at least one surface that extends laterally past the cannula's outer diameter. For instance, the cannula feature may comprise a crimp feature or a welded ferrule feature. The cannula feature further comprises a distal end and a proximal end. Accordingly, a portion, if not all, of the notch feature is disposed between the cannula feature's distal end and proximal end. The cannula may be used in any suitable assembly, including a catheter assembly comprising a needle capture mechanism.

### BACKGROUND OF THE INVENTION

This disclosure relates generally to cannulae and associated methods. More specifically, this disclosure discusses a cannula that comprises a cannula feature having a distal end and a proximal end, wherein at least a portion of a notch feature is defined in the cannula between the distal end and the proximal end. The cannula with the overlapping notch feature and cannula feature can be used in a variety of vascular access devices.

Generally, vascular access devices are used for communicating fluid with the vascular system of patients. For example, catheters are used for infusing fluid (e.g., saline solution, medicaments, and/or total parenteral nutrition) into a patient, withdrawing fluids (e.g., blood) from a patient, and/or monitoring various parameters of the patient's vascular system.

Intravenous (IV) catheter assemblies are among the various types of vascular access devices. Over-the-needle peripheral IV catheters are a common IV catheter configuration. As its name implies, an over-the-needle catheter is mounted over an introducer needle having a sharp distal tip. The introducer needle is generally a venipuncture needle coupled to a needle assembly that helps guide the needle and facilitates its cooperation with the catheter. At least the inner surface of the distal portion of the catheter tightly engages the outer surface of the needle to prevent peelback of the catheter and, thereby, to facilitate insertion of the catheter into the blood vessel. The catheter and the introducer needle are often assembled so that the sharp distal tip of the introducer needle extends beyond the distal tip of the catheter. Moreover, the catheter and needle are often assembled so that during insertion, the bevel of the needle faces up, away from the patient's skin. The catheter and introducer needle are generally inserted at a shallow angle through the patient's skin into a blood vessel.

In order to verify proper placement of the needle and/or the catheter in the blood vessel, the operator generally confirms that there is a “flashback” of blood into a flashback chamber associated with the needle assembly. Flashback generally entails the appearance of a small amount of blood, which exits the needle through an aperture and which is visible within the needle assembly or between the needle and the transparent catheter.

The operator may then withdraw the introducer needle from the catheter. In some cases, the introducer needle is withdrawn into a needle tip shield that extends over the needle tip and prevents accidental needle sticks. In general, a needle tip shield includes a casing, a sleeve, or another similar device that is designed to trap/capture the tip of the needle when the introducer needle is withdrawn from the catheter and the patient into the shielded position. After the needle is withdrawn, the catheter is left in place to provide intravenous access to the patient.

The separation of the introducer needle assembly from the catheter portions of the catheter assembly presents numerous potential hazards to the operator of the catheter device and others in the area. As indicated above, there is a risk of accidental needle sticks if the needle tip is not properly secured in a needle tip shield. Additionally, because the needle has been in contact with blood in the patient's vasculature, blood is often present on the needle's exterior as well as inside the lumen of the needle. As the needle is withdrawn from the catheter, there is a risk that this blood will drip from the needle tip or come into contact with other surfaces to expose people and equipment to blood. Additionally, where the needle comprises an aperture for flashback confirmation, this risk of blood exposure is often increased. For instance, because the aperture is adapted to allow blood to exit the lumen through the needle's sidewall, the aperture may also bypass or circumvent blood containment seals in the catheter assembly.

The present disclosure discusses a cannula with a cannula feature and an overlapping notch feature that allows flashback confirmation. The described cannula is configured to be moved and locked into a shielded position in a manner that significantly limits or prevents blood exposure and accidental sticks during use and after the cannula is withdrawn from the catheter assembly.

### BRIEF SUMMARY OF THE INVENTION

The present application relates to a cannula that is designed to overcome some of the limitations known in the art. In general, the cannula comprises a cannula feature that is at least partially overlapped by a notch feature. Because the notch feature at least partially extends into the cannula feature, both features can be disposed adjacent to the cannula's distal tip. Accordingly, a blood control seal that is configured to cover the cannula's distal tip and the notch feature can be relatively short. Similarly, a tip shielding device that is configured to capture the cannula's tip and cannula feature may be relatively short. Because the blood control seal and tip shielding device may be relatively short, they may fit within the bore of a catheter adapter until they are extracted for disposal.

The described cannula may have any suitable characteristic, including having a sharp, or relatively sharp, distal tip and an elongated tubular shaft that has a substantially constant outer diameter (“OD”). Additionally, the described cannula can be selected from a variety of cannula types, such as a venipuncture needle or another medical-grade, rigid tube having a sharpened distal tip. In some preferred implementations, the cannula comprises an introducer needle.

The cannula feature may comprise any type of cannula feature that has at least one surface that extends laterally past the cannula's OD. For instance, the cannula feature may comprise a crimp feature or a ferrule feature. In some presently preferred implementations, however, the cannula feature comprises a crimp feature. Such a crimp feature may be formed in any suitable manner, including by slightly crushing the cannula in a first axis and causing the cannula to bulge slightly in a second axis that runs orthogonally to the first axis. No matter the type of cannula feature used or the manner in which it is made, the cannula feature may also have any suitable characteristic. For instance, the cannula feature may comprise a distal end and a proximal end.

As mentioned, the cannula also comprises a notch feature that at least partially overlaps the cannula feature. The notch feature can comprise any suitable characteristic, including, size and shape. Furthermore, the notch feature can be defined in the cannula in any suitable manner, including through grinding, punching, stamping, electrical discharge machining, and laser cutting. However, because the cannula feature needs to remain strong in order to be used with a tip shielding device, methods, such as electrical discharge machining (“EDM”) and laser cutting, that remove a relatively small amount of the cannula's cross-sectional area may be preferred to methods, such as grinding, which can remove relatively large amounts of the cannula's cross-sectional sidewall area. Indeed, it may be preferable to remove as little of the cross-section area of the cannula's sidewall as is necessary to allow for flashback confirmation. In some instances the notch feature removes than about one half of the cross-sectional area of the cross-sectional area of the cannula's sidewall. In other instances, the notch feature removes less than about one fourth of the cannula's sidewall cross-sectional area.

As long as at least a portion of the notch feature is disposed between the cannula feature's distal and proximal ends, the notch feature can overlap virtually any portion of the cannula feature. In one example, the entire notch feature is disposed between the cannula feature's distal end and its proximal end. In another example, the notch feature extends across the cannula feature's distal end so that a first portion of the notch feature is disposed between the cannula feature's distal and proximal end and a second portion of the notch feature is disposed distal to the cannula feature's distal end. In still another example, the notch feature extends across the cannula feature's proximal end so that a first portion of the notch feature is disposed between the cannula feature's distal and proximal end and a second portion of the notch is disposed proximal to the cannula feature's proximal end. In yet another example, a first portion of the notch feature lies distal to the cannula feature's distal end and the notch feature extends across the cannula feature's distal and proximal ends so that a second portion of the notch feature lies proximal to the cannula feature's proximal end.

With respect to the cannula's bevel, the notch feature may have a variety of suitable orientations. For example, the notch feature may be defined in the cannula and/or the cannula feature so as to face in a direction that is substantially similar to, opposite to, perpendicular to, or otherwise offset from the direction in which the bevel of the cannula faces.

The described cannula may be used with any suitable system or device. In one example, the cannula may be used with any suitable catheter assembly, including an over-the-needle peripheral IV catheter assembly. In this example, the catheter assembly may further comprise a cannula capture mechanism. Such a capture mechanism may comprise any component that allows the cannula to become irreversibly trapped when the cannula is moved to the shielded position. For instance, the capture mechanism may comprise a proximal mating component and a distal mating component that respectively limit the cannula's proximal and distal movement respective to the capture mechanism when the cannula is in the shielded position.

In one example of a suitable proximal mating component, the capture mechanism comprises an aperture that is sized and shaped to allow the cannula, but not the laterally extending surface of the cannula feature, to translate proximally therethrough. Similarly, in an example of a suitable distal mating component, the capture mechanism comprises a needle tip capture mechanism, such as transverse barrier tips that are biased towards the cannula so as to limit the cannula's distal movement once the cannula has been retracted to the shielded position.

### BRIEF DESCRIPTION OF THE SEVERAL VIEWS OF THE DRAWINGS

In order that the manner in which the above-recited and other features and advantages of the invention are obtained and will be readily understood, a more particular description of the invention briefly described above will be rendered by reference to specific embodiments thereof, which are illustrated in the appended drawings. Understanding that these drawings depict only typical embodiments of the invention and are not, therefore, to be considered to be limiting of its scope, the invention will be described and explained with additional specificity and detail through the use of the accompanying drawings in which:

FIG. 1 illustrates a perspective view of a representative embodiment of a cannula comprising a crimp feature with an overlapping notch feature;

FIG. 2A illustrates a plan view of a representative embodiment of the cannula comprising a ferrule feature having an overlapping notch feature;

FIG. 2B illustrates a plan view of a representative embodiment of the cannula comprising one-way barbs that overlap the notch feature;

FIGS. 2C and 2D each illustrate plan views of a representative embodiment of the cannula comprising a protuberance and an overlapping notch feature;

FIG. 3A illustrates a perspective view of a representative embodiment of the cannula comprising a crimp feature and overlapping notch feature;

FIGS. 3B through 3D illustrate cross-sectional face views of some embodiments of the cannula in FIG. 3A taken at line 3-3;

FIG. 4A illustrates a plan view of a representative embodiment of the cannula in which the entire notch feature is disposed between a distal end and a proximal end of the crimp feature;

FIG. 4B illustrates a plan view of a representative embodiment of the cannula in which the notch feature extends across the distal end of the crimp feature;

FIG. 4C illustrates a plan view of a representative embodiment of the cannula in which the notch feature extends across the proximal end of the crimp feature;

FIG. 4D illustrates a plan view of a representative embodiment of the cannula in which the notch feature extends across both the distal end and the proximal end of the crimp feature;

FIG. 5 illustrates a cross sectional of a representative embodiment of the cannula in which the cannula's bevel and the notch feature have a similar orientation;

FIGS. 6A through 6C illustrate a representative embodiment of a method for making the cannula in which the crimp feature is formed before the notch feature;

FIGS. 7A through 7C illustrate a representative embodiment of a method for making the cannula in which the notch feature is formed before the crimp feature;

FIG. 8A illustrates a plan view of a representative embodiment in which the cannula is disposed in an unshielded position;

FIG. 8B illustrates a plan view of a representative embodiment in which the cannula is disposed in a shielded position;

FIG. 9A illustrates a plan view of a representative embodiment of a prior art cannula in which the notch feature is disposed distal to the crimp feature;

FIG. 9B illustrates a plan view of a representative embodiment of a hypothetical cannula in which the notch feature is disposed proximal to the crimp feature; and

FIG. 9C illustrates a plan view of a representative embodiment of the described cannula in which the entire notch feature is disposed between the distal end and the proximal end of the crimp feature.

### DETAILED DESCRIPTION OF THE INVENTION

The presently preferred embodiments of the described invention will be best understood by reference to the Figures, wherein like parts are designated by like numerals throughout. It will be readily understood that the components of the present invention, as generally described and illustrated in the accompanying Figures, could be arranged and designed in a wide variety of different configurations. Thus, the following more detailed description of some embodiments of the invention, as represented in the Figures, is not intended to limit the scope of the invention, as claimed, but is merely representative of some presently preferred embodiments of the invention.

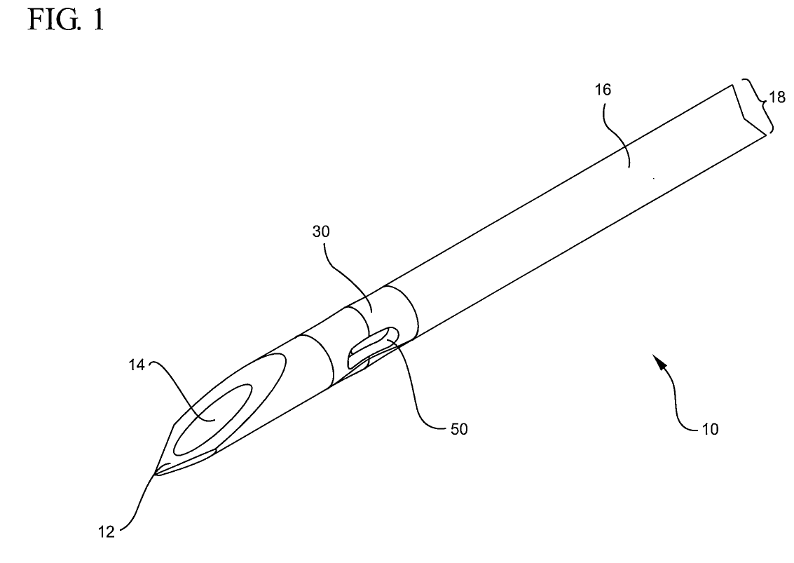

Generally, this disclosure discusses a cannula having an overlapping cannula feature and notch feature. While the described cannula can comprise any suitable component or characteristic, FIG. 1 illustrates a representative embodiment in which the cannula (e.g., needle 10) comprises a cannula feature (e.g., crimp feature 30) and a notch feature 50 that overlaps, or extends into, the cannula feature. To provide a better understanding of the cannula with overlapping notch and cannula features, the cannula, cannula feature, and notch feature are described below in greater detail.

The cannula may comprise virtually any rigid tube that both includes a sharpened distal tip and which is configured to puncture an animal's body, to access an intended space, and to draw off or to introduce fluid from or into the intended space. One example of a suitable cannula comprises a venipuncture needle. In this example, the venipuncture needle may include virtually any suitable venipuncture needle, including an introducer needle for use in an IV catheter assembly (e.g., an over-the-needle peripheral IV catheter assembly), a venous needle, an arterial needle, and the like. For simplicity, however, the cannula with a cannula feature and overlapping notch feature is described below with reference to an introducer needle.

The introducer needle may have any component that is suitable for use with an IV catheter assembly. For instance, FIG. 1 shows a representative embodiment in which the introducer needle 10 comprises a sharpened distal tip 12, an inner lumen 14, and an elongated tubular shaft 16 having a substantially constant outer diameter (“OD”) 18. Moreover, each component of the needle may have any suitable characteristic. For example, the distal tip of the needle may comprise a standard bevel, a short bevel, a true short bevel, a bias grind point, a vet point, a lancet point, a deflected point (anti-coring), or another suitable known or novel needle point. Additionally, the lumen and elongated tubular shaft may be any suitable size. For example, the needle may be any suitable length or any suitable gauge (e.g., from about a 7 to about a 33 on the Stubs scale) that allows it to be used as the introducer needle in an IV assembly.

As previously mentioned, cannula further comprises a cannula feature. The cannula feature may serve any suitable purpose. For example, the cannula feature can provide the cannula with a proximal engagement, or a surface that is configured to be captured by a cannula feature capture mechanism (described hereinafter). Accordingly, the cannula feature can help limit the cannula's proximal movement once the cannula is retracted into a shielding device.

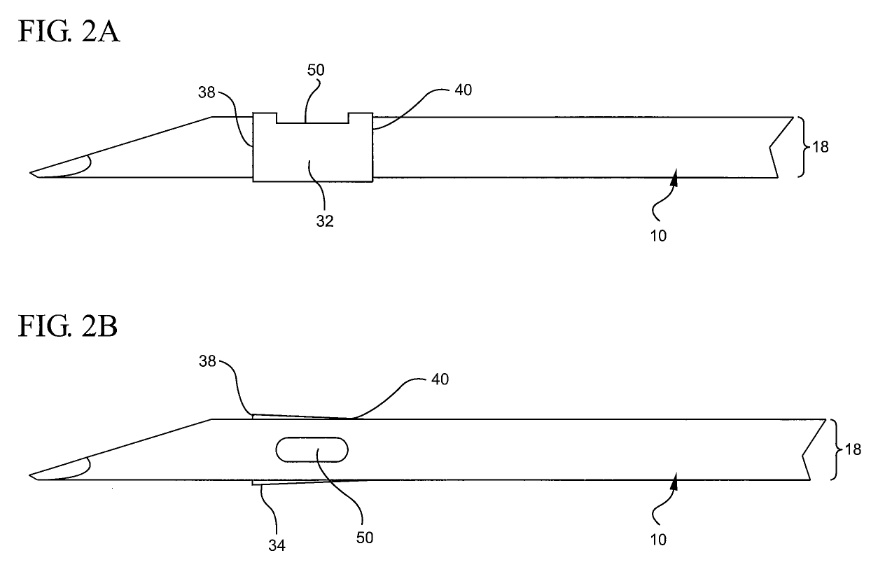

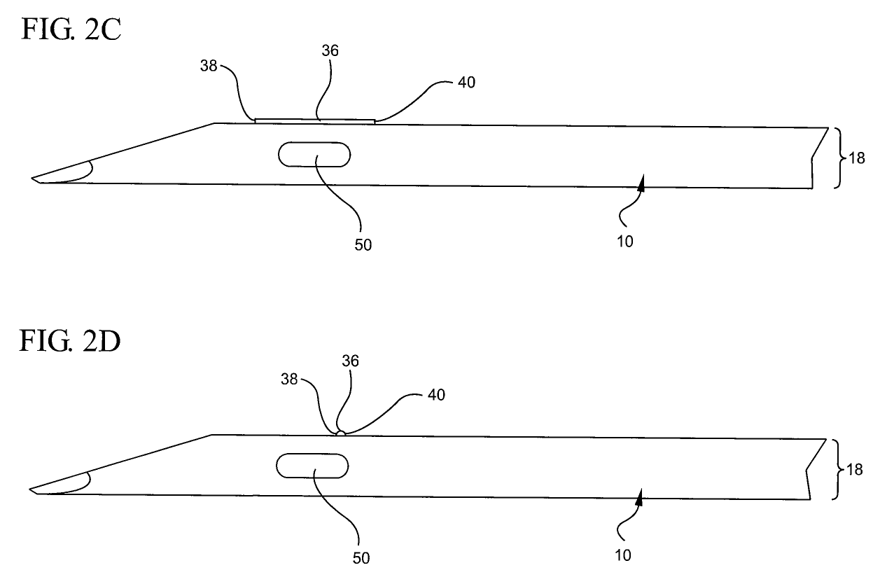

The cannula feature may comprise any suitable component that has at least one surface that extends laterally past the needle's OD and which is adapted to act as a proximal engagement. Some examples of suitable cannula features comprise one or more crimp features, ferrule features, protuberances, one-way barbs, laterally extending members (e.g., a ring), and other components that have a laterally extending surface. By way of non-limiting illustration, FIG. 1 shows an embodiment in which the cannula feature comprises a crimp feature 30. Similarly, FIG. 2A shows a representative embodiment in which the cannula feature comprises a welded ferrule feature 32. FIG. 2B illustrates a representative embodiment in which the cannula feature comprises at least one one-way barb 34. Finally, FIGS. 2C and 2D illustrate some embodiments in which the cannula feature comprises a protuberance 36.

The cannula feature may comprise any suitable component or characteristic that allows it to be used with a catheter assembly and to at least prevent the cannula from being completely extracted proximally through a capture mechanism. By way of example, FIGS. 1 through 2D shown the cannula feature (e.g., features 30, 32, 34, and 36) comprises a distal end 38 and a proximal end 40. Specifically, FIGS. 1 through 2D show that the distal end 38 of the cannula feature (i.e., 30, 32, 34, and 36) is the point at which the distal-most portion of the cannula feature extends laterally from the needle's OD 18. Likewise, FIGS. 1 through 2D show that the proximal end 40 of the cannula feature (i.e., 30, 32, 34, and 36) is the point at which the proximal-most portion of the cannula feature extends laterally from the needle's OD 18.

As mentioned above, the needle also defines a notch feature that at least partially overlaps the cannula feature. In other words, at least a portion of the notch feature is disposed between the cannula feature's distal end and its proximal end. This notch feature may serve several purposes, including acting as an aperture through which blood can exit the inner lumen of the needle to provide flashback confirmation when the needle is properly placed in a patient's vasculature.

The notch feature can have any suitable characteristic that allows it to provide the needle with flashback confirmation. In one example, from a face view of the notch feature, the notch feature can be any suitable shape, including a shape that is substantially elliptical, square, rectangular, triangular, circular, polygonyl, irregular, etc. For instance, FIG. 2D illustrates an embodiment in which the notch feature 50 is substantially elliptical.

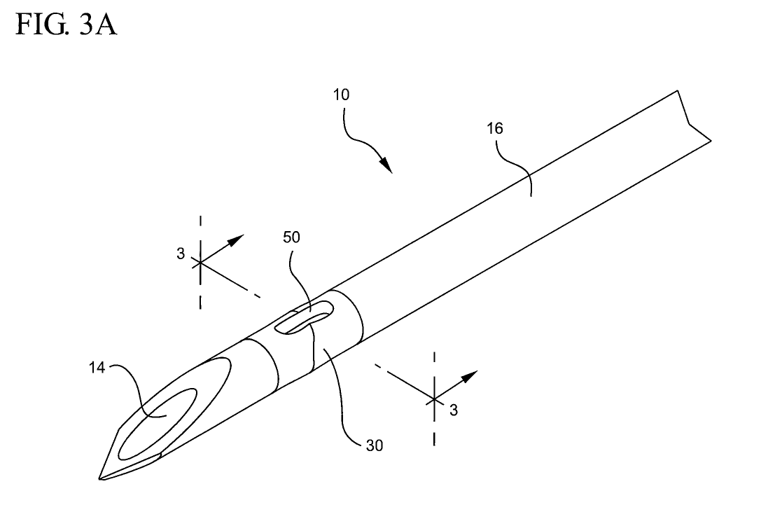

In another example of a suitable characteristic, the notch feature may remove any amount of a cross-sectional area of the needle's sidewall that allows the notch feature to provide the needle with flashback confirmation while still allowing the cannula feature to have sufficient structural integrity to fulfill its intended purposes. By way of illustration, FIGS. 3B through 3D show cross-sectional views of various embodiments of the needle 10 in FIG. 3A, cut along line 3-3. Specifically, FIG. 3B shows that, in some embodiments, the notch feature 50 removes about one half of the cross-sectional area 52 of the sidewall 20 of the needle 10. However, FIG. 3C shows that, in some presently preferred embodiments, the notch feature 50 removes less than about one half of the cross-sectional area 52 of the needle's sidewall 20. FIG. 3D illustrates that, in still other preferred embodiments, the notch feature 50 removes less than about one fourth of the cross-sectional area 52 of the needle's sidewall 20.

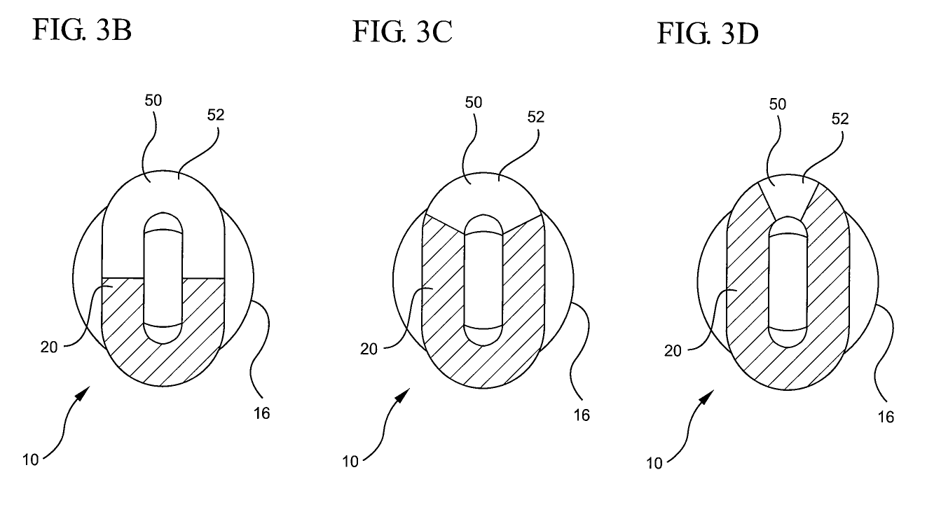

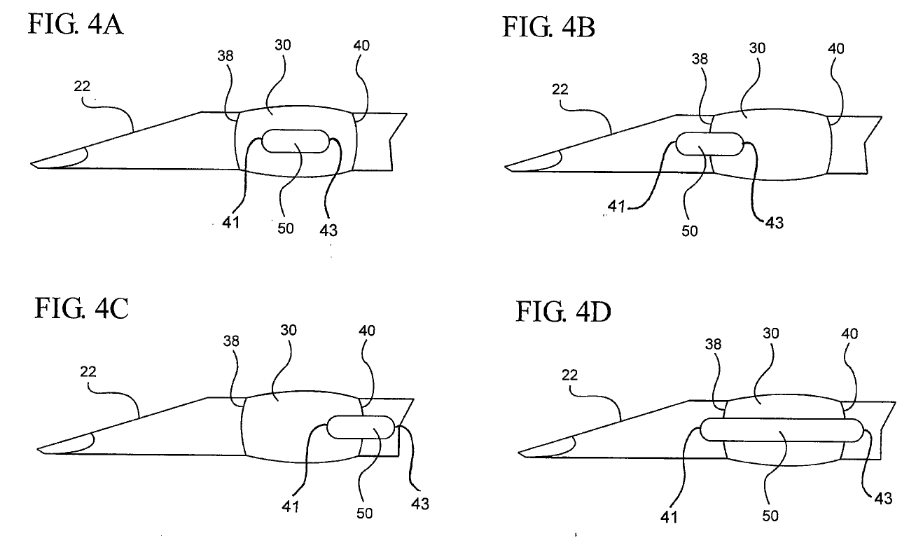

The notch feature may overlap the cannula feature in any suitable manner that allows at least a portion of the notch feature to be disposed between the cannula feature's distal end and proximal end. In some embodiments, a distal-most edge of the notch feature is disposed at or proximal to the distal end of the cannula feature. By way of illustration, FIG. 4A shows a representative embodiment in which the entire notch feature 50 is disposed between the cannula feature's distal end 38 and proximal end 40. More specifically, in FIG. 4A, the notch feature's distal-most edge 41 is disposed proximal to the cannula feature's distal end 38 and the proximal-most edge 43 of the notch feature 50 is disposed distal to the cannula feature's proximal end 40.

In other embodiments, only a portion of the notch feature is disposed between the distal end and the proximal end of the cannula feature. In one example, FIG. 4B shows an embodiment in which the distal-most edge 41 of the notch feature 50 is disposed distal to the cannula feature's distal end 38 while the notch feature's proximal-most edge 43 is disposed proximal to the cannula feature's distal end 38. In another example, FIG. 4C shows an embodiment in which the proximal-most edge 41 of the notch feature 50 is disposed distal to the cannula feature's proximal end 40 while the notch feature's proximal-most edge 43 is disposed proximally past the cannula feature's proximal end 40. In a final example, FIG. 4D shows an embodiment in which the distal-most edge 41 of the notch feature 50 is disposed distal to the cannula feature's distal end 38 while the notch feature's proximal-most edge 43 is disposed proximally past the proximal end 40 of the cannula feature (e.g., crimp feature 30).

The notch feature may also have any orientation with respect to the needle's bevel that allows the notch feature to fulfill its intended purposes. In some embodiments, the notch feature is defined in the cannula so as to be offset in orientation from the direction in which the needle's bevel faces. In some instances in which the notch feature is defined in the needle so as to be offset from the direction of the bevel, the notch feature may allow for faster flashback confirmation in instances in which the user's line of sight includes the side of the needle in which the notch is defined but does not include the side of the needle from which the bevel faces.

Where the notch feature is offset from the direction of the needle's bevel, the notch feature can be offset in any suitable manner. For instance, the notch feature can be defined in the needle so as to face in an opposite direction to, a perpendicular direction to, or in another suitable direction with respect to the direction in which needle's bevel faces. By way of example, FIG. 4D shows an embodiment in which the notch feature 50 faces in a direction that is offset from the needle's bevel 22 so that the notch feature is oriented substantially perpendicular to the direction in which the needle's bevel 22 faces. In other words, the notch feature 50 in FIG. 4D is disposed substantially perpendicular to a plane (not shown) that symmetrically bisects the needle 10 along its length.

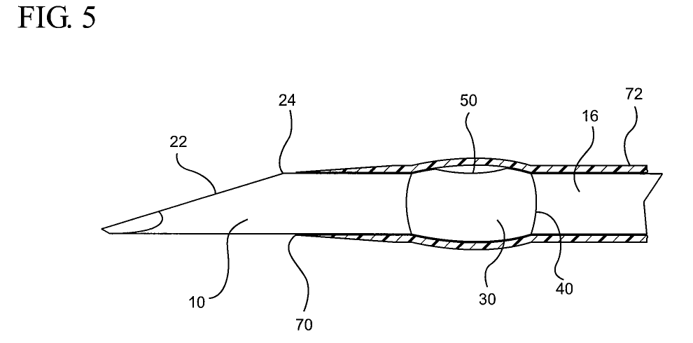

In other embodiments, the notch feature is defined in the cannula so as to face in substantially the same direction as the needle's bevel. By way of illustration, FIG. 5 illustrates a representative embodiment in which the notch feature 50 faces in substantially the same direction as the needle's bevel 22. Said differently, the notch feature in FIG. 5 is oriented so that a plane (not shown) that symmetrically bisects the length of the needle 10 also symmetrically bisects the notch feature 50. Because, in some instances, flashback confirmation may be visualized slightly sooner where the needle's bevel and the notch feature face in substantially similar directions, such an embodiment may be preferred in some circumstances.

FIG. 5 also illustrates that the notch feature 50 and the proximal end 40 of the cannula feature (crimp feature 30) may be disposed any distance (x) from the proximal-most end 24 of the needle's bevel 22 that allows the needle to be used as intended and that allows the distal tip 70 of a catheter 72 to form a tight seal around the needle's outer surface 26. In some preferred embodiments, however, the notch feature and the proximal end of the cannula feature are disposed as close as possible to the proximal-most end of the bevel. Indeed, as the notch feature and the proximal end of the cannula feature are disposed closer to the needle's distal tip, the necessary length of the tip shielding device and blood control seal, as are known in the art, may be shorter and still fulfill their intended purposes.

In addition to the previously mentioned embodiments, the described needle having a cannula feature with an overlapping notch feature may be varied in any suitable manner. In one example, the needle comprises more than one notch feature. In this example, the needle can comprise any suitable number of notch features, including 2, 3, or more. Furthermore, in this example, the additional notch features may, but do not necessarily, overlap the cannula feature.

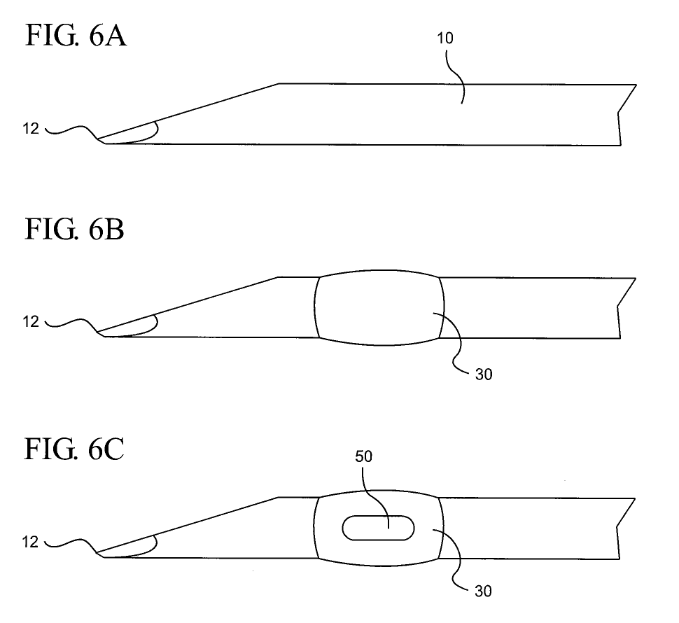

The described needle may be made in any suitable manner. In one example, the cannula feature is formed before the notch feature. By way of illustration, FIG. 6A shows that this method may begin by providing a needle 10 with a sharpened distal tip 12. FIG. 6B shows that method continues by forming a cannula feature, such as the crimp feature 30, in any suitable manner. For instance, the crimp feature may be formed by upsetting the needle or slightly crushing the needle in a first axis to cause the needle to bulge slightly in a second axis that runs orthogonal to the first axis.

Following the formation of the cannula feature, FIG. 6C shows that the notch feature 50 is formed. While the notch feature may be formed in any suitable manner, including, but not limited to, grinding, stamping, punching, EDM, and/or laser cutting, the notch feature is preferably formed through a method that removes as little of the needle's cross-sectional area as possible while still allowing suitable flashback confirmation. Indeed, because grinding and some other techniques may weaken the cannula feature by removing about one half of the needle's cross-sectional area, EDM and/or laser cutting are preferable in certain circumstances.

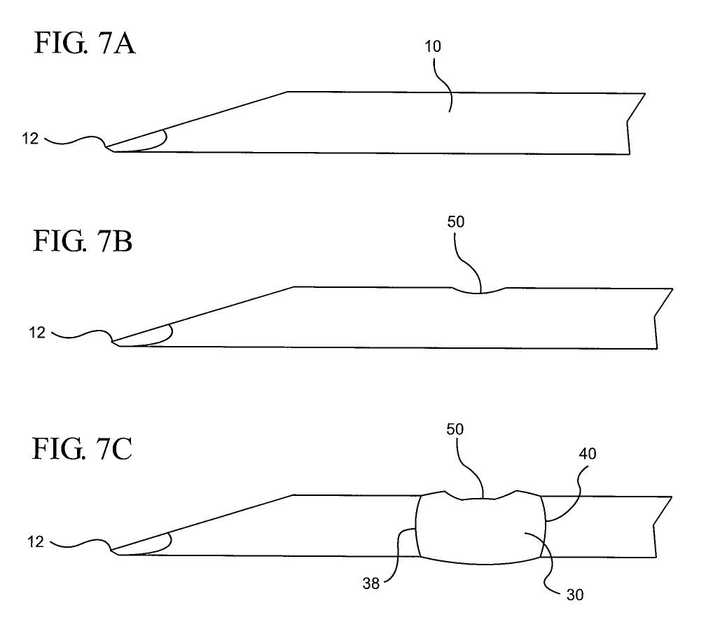

In another example of a suitable method for forming the described needle, the notch feature is formed before the cannula feature. For instance, FIG. 7A shows that such a method begins by providing a needle 10 with a sharpened distal tip 12. FIG. 7B shows the method continues by forming the notch feature 50 in the needle 10. Finally, FIG. 7C shows the crimp feature 30 is formed so that at least a portion of the notch feature 50 is disposed between the cannula feature's distal end 38 and proximal end 40.

The described needle can be used with any suitable device or assembly. In one previously mentioned example, the needle is used with a catheter assembly. In this example, the catheter assembly may comprise any suitable component, including a needle capture mechanism that is capable of trapping the needle in the shielded position and limiting the needle's distal and proximal movement respective to the capture mechanism. Although a suitable capture mechanism may comprise any suitable component, in some embodiments, the needle capture mechanism comprises a distal mating component and a proximal mating component.

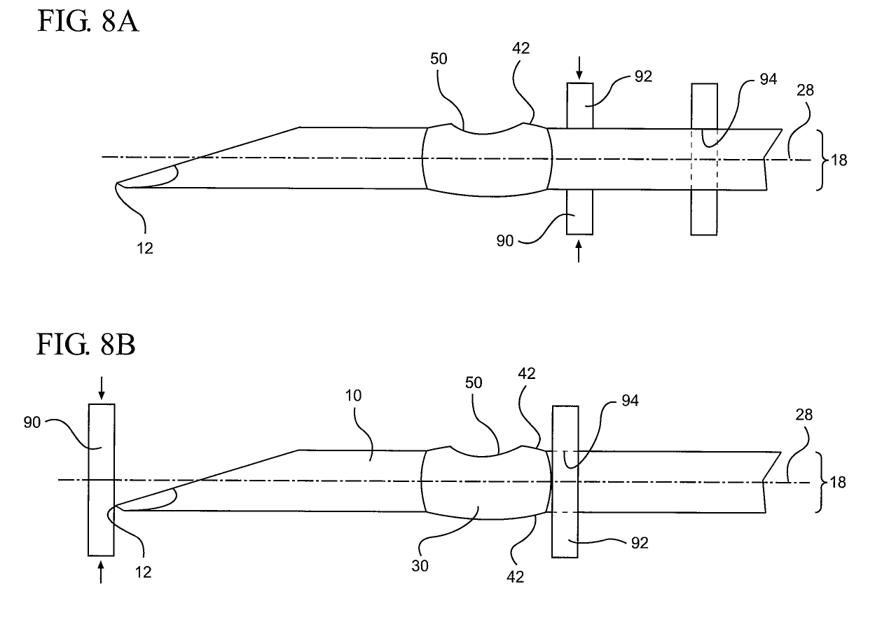

In such embodiments, the distal mating component may be any structure that is adapted to contact the needle and limit the needle's distal movement once the needle has been moved to a shielded position. For instance, the distal mating component may comprise any suitable needle tip capture device or cannula feature capture device that is configured to limit the needle's distal movement after the needle is retracted to the shielded position. By way of illustration, FIG. 8A shows a representative embodiment in which the distal mating component comprises one or more transverse tip barriers 90 (e.g., portions of a split housing) that are biased towards the longitudinal axis 28 of the needle 10. Accordingly, FIG. 8B illustrates that when the distal tip 12 of the needle 10 is retracted proximally past the transverse tip barriers 90, the tip barriers 90 move into a position that blocks the distal movement of the needle's distal tip 12. In this manner, the tip barriers irreversibly lock the needle in the shielded position and prevent the needle from reemerging and causing unintentional sticking.

The proximal mating component of the capture mechanism may comprise any component that is adapted to contact the needle feature's proximal engagement (e.g., the proximal surface 42 of the needle feature 30 in FIG. 8A that extends laterally past the needle's OD 18) and limit the feature's proximal movement once the needle has been moved to a shielded position. For instance, the proximal mating component may be any suitable component that has an opening sized to allow the needle, but not the laterally extending surface(s) of the needle feature, to pass therethrough. By way of illustration, FIG. 8A illustrates a representative embodiment in which the proximal mating component comprises a rigid washer 92. FIG. 8B illustrates that when the needle 10 is moved from the unshielded position (shown in FIG. 8A) to the shielded position, the proximal surfaces 42 of the needle feature 30 that extend laterally past the needle's OD 18 are too large to pass through the inner diameter 94 of the washer 92.

Thus, once the needle has been moved in between the capture mechanism's distal and proximal mating components (e.g., transverse tip barriers 90 and washer 92, respectively), the sharp distal tip of the needle becomes irreversibly trapped. As a result, the needle is locked in the shielded position for proper disposal.

In addition to the aforementioned benefits, the described needle may offer several additional benefits and advantages. For instance, the described needle may be used with a shorter tip shielding device than may certain conventional needles of a comparable size in which a notch feature and cannula feature are non-overlapping. As previously stated, because the cannula feature and the notch feature in the described needle at least partially overlap each other, the distance between the needle's distal tip and the proximal end of the cannula feature may be shorter than in certain conventional needle's. Accordingly, because a tip shielding device with transverse tip barriers must be at least as long as the distance from the cannula's distal tip to the proximal side of the cannula feature, the tip shielding device for the described needle can be shorter than comparable tip shielding devices for use with some conventional needles in which the notch feature and the crimp feature are non-overlapping.

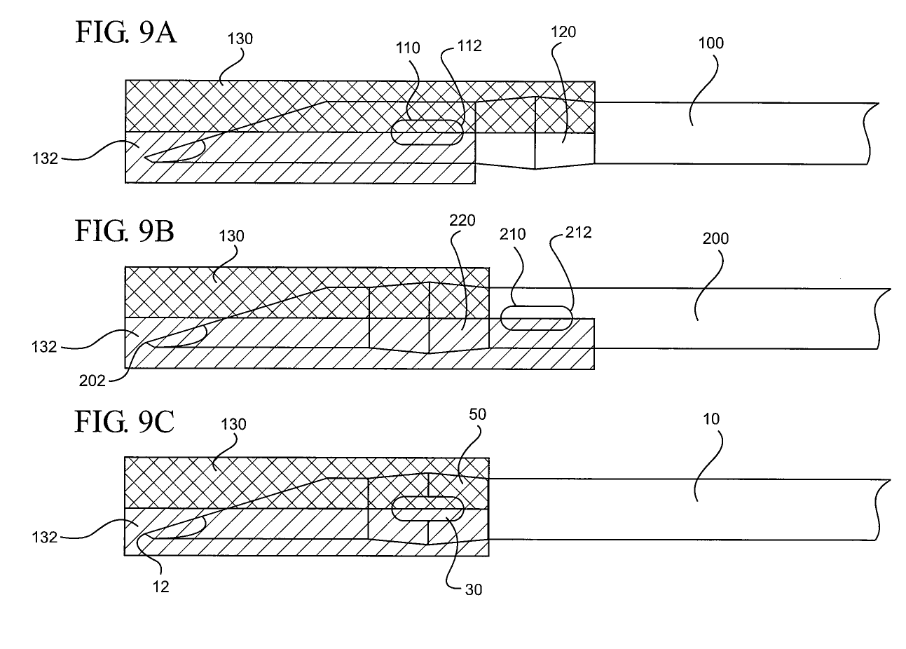

By way of illustration, FIGS. 9A illustrates an example of a conventional needle 100 in which the notch feature 110 is disposed sufficiently distal to the cannula feature 120 so as to not overlap the cannula feature 120. In this example, FIG. 9A illustrates that the tip shield device must be relatively long, as illustrated by the cross-hatched section 130. Additionally, FIG. 9A shows that in the prior art needle 100, the blood control seal, which need only extend to the proximal end 112 of the notch feature 110, may be relatively short, as is illustrated by the hatched section 132.

In contrast, FIG. 9B illustrates a hypothetical needle 200 in which the notch feature 210 is disposed proximal to the cannula feature 220 so as to not overlap with the cannula feature 220. Accordingly, FIG. 9B shows that because the notch feature 210 in the needle 200 is not disposed between the needle's distal tip 202 and the cannula feature 220, the cannula feature 220 may be disposed relatively close to the distal tip 202 and, thereby, allow the tip shield device to be relatively short, as is illustrated by the cross-hatched portion 130. However, FIG. 9B shows that in order to extend past the cannula feature 220 to the proximal end 212 of the notch feature 210, the blood control seal for such a needle 200 must be relatively long, as is illustrated by the hatched section 132.

Finally, FIG. 9C illustrates a representative embodiment of the described needle 10. In so doing, FIG. 9C shows that because the notch feature 30 and the cannula feature 50 overlap each other, both features 30 and 50 may be disposed relatively close the cannula's distal tip 12. As a result, FIG. 9C shows that both the tip shield device and the blood control seal may both be relatively short, as illustrated by the cross-hatched section 130 and the hatched section 132, respectively. Because both the blood control seal and the tip shield device of the described needle can be relatively short, the blood control seal and the tip shield device may both be disposed within the bore of a catheter adapter (not shown) when the needle is in the unshielded position.

The present invention may be embodied in other specific forms without departing from its structures, methods, or other essential characteristics as broadly described herein and claimed hereinafter. All of the described embodiments and examples are to be considered in any and all respects as illustrative only, and not restrictive. The scope of the invention is, therefore, indicated by the appended claims, rather than by the foregoing description. All changes that come within the meaning and range of equivalency of the claims are to be embraced within their scope.

1. A cannula, comprising:
a shaft having an outer diameter;
a cannula feature comprising a surface that extends laterally past the shaft's outer diameter, wherein the cannula feature further comprises a distal end and a proximal end; and
a notch feature defining an opening in the shaft, wherein a distal-most edge of the notch feature is disposed at or proximal to the distal end of the cannula feature such that at least a portion of the cannula feature and the notch feature overlap and such that the opening in the shaft extends through and penetrates at least a portion of the cannula feature.

2. The cannula of claim 1, wherein the cannula feature comprises a crimp feature.

3. The cannula of claim 1, wherein the notch is disposed between the distal end and the proximal end of the cannula feature.

4. The cannula of claim 1, wherein a proximal-most edge of the notch feature extends proximally past the proximal end of the cannula feature.

5. The cannula of claim 1, wherein the notch feature removes less than about one fourth of a cross-sectional area of a sidewall of the shaft.

6. The cannula of claim 1, wherein the notch feature is defined in the shaft so as to be offset in orientation from a direction in which a beveled surface of a distal tip of the shaft faces.

7. The cannula of claim 1, wherein the cannula feature is selected from a one-way barb, a protuberance, and a ferrule.

8. A catheter assembly, comprising:
a catheter assembly having a needle capture mechanism; and
a needle comprising a notch feature defining an opening in the needle and having a crimp feature comprising a distal end and a proximal end,
wherein a distal-most edge of the notch feature is disposed at or proximal to the distal end of the crimp feature such that at least a portion of the cannula feature and the notch feature overlap and such that the opening in the shaft extends through and penetrates at least a portion of the crimp feature.

9. The assembly of claim 8, wherein the entire notch feature is disposed between the distal end and the proximal end of the crimp feature.

10. The assembly of claim 8, wherein a proximal-most edge of the notch feature extends proximally past the proximal end of the crimp feature.

11. The assembly of claim 8, wherein the notch feature is defined in the needle so as to be offset in orientation from a direction in which a beveled surface of a distal tip of the needle faces.

12. The assembly of claim 8, wherein the notch feature removes less than about one fourth of a cross-sectional area of a sidewall of the needle.

13. The assembly of claim 8, wherein the crimp feature is selected from a one-way barb, a protuberance, and a ferrule.

14. The assembly of claim 13, wherein the needle capture mechanism further comprises a needle tip capture device.

15. A method for making a needle, the method comprising:
providing a needle;
forming a crimp feature on the needle, the crimp feature having a distal end and a proximal end; and
defining a notch feature in the needle the notch forming an opening in the needle,
wherein a distal-most edge of the notch feature is disposed at or proximal to the distal end of the crimp feature such that at least a portion of the crimp feature and the notch feature overlap and such that the opening in the shaft penetrates at least a portion of the crimp feature.

16. The method of claim 15, wherein the entire notch feature is disposed between the distal end and the proximal end of the crimp feature.

17. The method of claim 15, wherein a proximal-most edge of the notch feature extends proximally past the proximal end of the crimp feature.

18. The method of claim 15, wherein the notch feature is defined in the needle so as to be offset in orientation from a direction in which a beveled surface of a distal tip of the needle faces.

19. The method of claim 15, wherein the notch feature is formed before the crimp feature.

20. The method of claim 15, wherein the crimp feature is formed before the notch feature.
